(19) United States
(12) Patent Application Publication
(10) Pub. No.: US 2024/0366309 A1
(43) Pub. Date: Nov. 7, 2024

(54) SYSTEM AND METHODS FOR NAVIGATION OF CATHETERS WITHIN VESSELS

(71) Applicant: MG Stroke Analytics Inc., Calgary (CA)

(72) Inventors: Mayank GOYAL, Calgary (CA); Johanna OSPEL, Calgary (CA)

(21) Appl. No.: 18/310,035

(22) Filed: May 1, 2023

(51) Int. Cl. A61B 34/20; A61B 90/00; A61M 1/36; A61M 25/00; A61M 25/01

(52) U.S. Cl. CPC A61B 34/20; A61B 90/39; A61M 1/3672; A61M 25/0021; A61M 25/0108; A61B 2090/3966; A61M 2025/0042

(57) ABSTRACT

Systems and methods for navigation of catheters through vessels including intracranial venous vessel access are described. In addition, systems and methods for the treatment of vasculature conditions including aspiration from the cerebral venous sinus are described. Generally, the system includes a co-axial combination of a microwire, a support catheter having an oval section and an aspiration catheter. Methods of advancing the system through the vasculature including the venous vasculature with narrow sections are also described.

### FIELD

Systems and methods for navigation of catheters through vessels including intracranial venous vessel access are described. In addition, systems and methods for the treatment of vasculature conditions including aspiration from the cerebral venous sinus are described. Generally, the system includes a co-axial combination of a microwire, a support catheter having an oval section and an aspiration catheter. Methods of advancing the system through the vasculature including the venous vasculature with narrow sections are also described.

### BACKGROUND

Cerebral venous thrombosis (CVT) refers to occlusion of venous channels in the cranial cavity. These can generally be sub-characterized as dural venous sinus thrombosis (DVST), cortical vein thrombosis and deep cerebral vein thrombosis. These conditions often co-exist and the clinical presentation can be very similar and nonspecific. Furthermore, the diagnostic imaging features can be subtle.

DVST is the most common condition. DVST is most likely to affect women on the contraceptive pill. However, other risk factors include lifestyle factors, other hormonal factors, drugs, anatomical/trauma and disease conditions. As such, more specific risk factors can include smoking, pregnancy, puerperium, steroids, and hyperthyroidism, prothrombotic haematological conditions including protein S deficiency and polycythaemia, COVID-19 and COVID-19 vaccination, local factors including skull abnormalities, infections (especially mastoid sinus—dural sinus occlusive disease) and head injury (especially skull fractures that extends to a dural venous sinus) and systemic illness including dehydration, sepsis, malignancy and connective tissue disorders. DVST may also be from idiopathic causes.

Presentation is variable and can range from asymptomatic to coma and death. Typically, patients complain of headache, nausea, and vomiting. Neurological deficits are variable. Since DVST obstructs the brain's venous outflow, venous pressure increases, backpressure builds up and cerebral swelling may occur. The subsequent venous hypertension can lead to edema and haemorrhage.

Once diagnosed, treatment can be challenging. Presently, systemic anticoagulation (e.g. heparin and warfarin) is still the first-line treatment for dural venous sinus thrombosis. Anticoagulation is usually required even in the setting of venous hemorrhage.

Interventional management includes microcatheter thrombolysis or thrombectomy (mechanical removal of thrombus material from the cerebral veins and sinuses using microcatheters and other endovascular tools). As discussed in greater detail below, microcatheter intervention can be challenging with currently available catheter systems. That is, due to the relative rarity of venous thrombosis (as compared to arterial ischemic stroke), no catheters dedicated to venous thrombectomy are available, and physicians must use catheter systems designed and/or engineered for cerebral artery access and thrombectomy.

However, the specifics of CVT and cranial venous anatomy both have particular features that can limit the effectiveness of arterial access/thrombectomy catheters in the venous system.

Structurally, arterial access catheters are characterized by a maximum outside diameter (OD) of about 8 French (2.67 mm) (and usually much smaller). That is, due to the characteristics of arterial ischemic stroke including the progressive narrowing of the distal arterial vessels and the blood pressure of the arterial system, the maximum diameter of larger distal access catheters (DACs) is about 8 French. As a result, arterial recanalization procedures typically use various combinations of bi-, tri- and quadra-axial catheter systems to progressively gain access to more distal regions of the cerebral arteries where the thrombosis may be located.

For reference, Table 1 shows the comparison of French, metric and imperial units used in catheters.

| French | Circumference | Diameter | Diameter |
|---|---|---|---|
| Gauge | (mm) | (mm) | (inches) |
| 3 | 3.14 | 1 | 0.039 |
| 4 | 4.19 | 1.333 | 0.053 |
| 5 | 5.24 | 1.667 | 0.066 |
| 6 | 6.28 | 2 | 0.079 |
| 7 | 7.33 | 2.333 | 0.092 |
| 8 | 8.34 | 2.667 | 0.105 |
| 9 | 9.42 | 3 | 0.118 |
| 10 | 10.47 | 3.333 | 0.131 |
| 11 | 11.52 | 3.667 | 0.144 |
| 12 | 12.57 | 4 | 0.158 |
| 13 | 13.61 | 4.333 | 0.170 |
| 14 | 14.66 | 4.667 | 0.184 |
| 15 | 15.71 | 5 | 0.197 |
| 16 | 16.76 | 5.333 | 0.210 |
| 17 | 17.81 | 5.667 | 0.223 |
| 18 | 18.85 | 6 | 0.236 |
| 19 | 19.90 | 6.333 | 0.249 |
| 20 | 20.94 | 6.667 | 0.263 |
| 22 | 23.04 | 7.333 | 0.288 |
| 24 | 25.13 | 8 | 0.315 |
| 26 | 27.23 | 8.667 | 0.341 |
| 28 | 29.32 | 9.333 | 0.367 |
| 30 | 31.42 | 10 | 0.393 |
| 32 | 33.51 | 10.667 | 0.419 |
| 34 | 35.60 | 11.333 | 0.445 |

The design of arterial access catheters is specific to the arterial anatomy and various features and properties are incorporated into arterial catheters to enable their successful progress into the cranial vasculature to conduct various recanalization procedures.

In contrast, the cranial venous system has its own specific anatomical features that create unique problems to the navigation of catheters into the venous vasculature. Similarly, DVST is morphologically dissimilar to arterial thrombosis.

For example, as shown in schematically in FIGS. 1 and 1B, the dural sinus has a larger diameter (around 0.8-1 cm) and DVST may present as a substantial compromise of this vessel along a length of the dural sinus as much as 20 cm. Moreover, as the DVST thrombus forms in situ, the DVST thrombus will often present as a compromise in the lumen of the dural sinus (as opposed to complete blockage) along this distance. The clot may also extend into the cortical veins and/or exist at the junction between the dural sinus and the cortical veins.

The residual lumen may be as little as 1 mm. Hence, placement of arterial access catheters within the dural sinus and aspiration of a clot using an 8 French (2.67 mm) distal access catheter may simply create a relatively small channel through the clot which does not significantly reduce the clot burden or alleviate the condition as shown in FIGS. 1C and 1D. That is, a clot Y in the superior sagittal sinus (SSS) may have a diameter of about 0.8-1 cm and an aspiration catheter AC has a diameter of 2.67 mm such that only a relatively small hole H is created in the clot Y when aspiration is applied to the clot Y.

Arachnoid granulations also present an access and navigation challenge to catheter access. As shown in FIG. 1, arachnoid granulations (AGs) exist as bulges that deform the superior sagittal sinus (SSS) that can cause a substantial obstruction to the progress of a catheter as a result of this localized narrowing.

Accordingly, there has been a need for catheter systems specifically designed for and having the functionality to be effectively positioned within the cerebral venous vasculature to enable DVST treatment and that are able to navigate unique features of the cerebral venous system.

### SUMMARY

A catheter system to aid in navigating a catheter through a vessel having a narrow section or tortuous section of a patient's vasculature is described, the catheter system having an outer catheter (OC) having an OC distal end having an OC distal end inner diameter (ID) and inner perimeter (IP); a support catheter (SC) having an SC distal tip end, an SC proximal end and an SC expanded section adjacent the SC distal tip end, the SC expanded section having a distal taper, a proximal taper and an outer perimeter (OP), the OP substantially corresponding to the IP of the OC the expanded section for flattening the OC distal end during catheter placement.

In various embodiments:
the expanded section has an oval cross-section.
the OC is an aspiration catheter (AC).
the SC expanded section has a major axis and minor axis and the major:minor ratio is 1.5:1.
the distal taper is 4-12 cm.
the proximal taper is 4-12 cm.
the expanded section is 4-12 cm.
the expanded section has a longitudinal axis and a distal end of the expanded section can twist about the longitudinal axis relative to a proximal end of the expanded section.
the distal tip of the AC includes one or more radio-opaque dot markers adjacent the distal tip.
the dot markers are at least two dot markers forming a non-continuous band around the circumference of the aspiration catheter adjacent the distal tip.

In another embodiment, a catheter system to aid in navigating a catheter through a vessel having a narrow section or tortuous section of a patient's vasculature is described having, an outer catheter (OC) having an OC distal end having an OC distal end inner diameter (ID) and inner perimeter (IP); a support catheter (SC) having an SC distal tip end, an SC proximal end and an SC expanded oval section adjacent the SC distal tip end, the SC expanded section having a distal taper, a proximal taper and an outer perimeter (OP), the OP substantially corresponding to the IP of the OC the expanded section for flattening the OC distal end during catheter placement; and, one or more radio-opaque dot markers adjacent the distal tip of the OC.

In various embodiments, the distal taper is 4-12 cm, the proximal taper is 4-12 cm and/or the expanded section is 4-12 cm.

In another aspect, a method of advancing a catheter system as described herein through vessels of a patient having a narrowed section is described, comprising the steps of: a) introducing the catheter system into the patient at an access point; b) advancing an SC and OC from the access point towards the narrowed section; and c) engaging the OC over the expanded section to distort the OC distal tip to an oval shape and pushing the supported OC distal tip past the narrowing.

The method may also include the step of collecting and cleaning recovered blood and reintroducing recovered and cleaned blood back to the patient.

### BRIEF DESCRIPTION OF THE DRAWINGS

Various objects, features and advantages of the disclosure will be apparent from the following description of particular embodiments as illustrated in the accompanying drawings. The drawings are not necessarily to scale, emphasis instead being placed upon illustrating the principles of various embodiments of the invention. Similar reference numerals indicate similar components.

FIGS. 1, 1A and 1B are schematic sketches of brain vascular anatomy showing features of the venous vasculature and a representative DVST thrombus (FIG. 1) and an arachnoid granulation (AG) protruding against the superior sagittal sinus (SSS) (FIG. 1A).

FIGS. 1C and 1D are schematic diagrams showing the problem of aspirating a large diameter clot within a large vessel with a small diameter aspiration catheter.

FIG. 2 is a sketch of a typical microwire, microcatheter and distal access catheter that may used for arterial recanalization procedures in accordance with the prior art and illustrating the problem of separation between internal microcatheters/wires and outer catheters that can bind or be problematic to advance through regions of higher tortuosity and/or narrowing in the vasculature.

FIG. 2A is a sketch of the venous vasculature illustrating the problem of advancing a DAC over a microcatheter in a region of high tortuosity.

FIG. 3 is a sketch of a venous access system in accordance with one embodiment.

FIGS. 3A-3F are perspective views of a catheter system and a process of navigation.

FIGS. 3G and 3H are perspective views of an oval support section of a catheter system and a distal tip of a support catheter.

FIG. 4A is a sketch illustrating the problem of navigating a catheter past a narrow section of a vasculature.

FIG. 4B is a sketch illustrating how an oval section may navigate through a narrow section of a vasculature.

FIGS. 5A and 5B are schematic diagrams showing radio-opaque markers in accordance with the prior art and present disclosure.

### DETAILED DESCRIPTION

With reference to the figures, systems and methods for accessing cerebral venous thrombi are described.

Key structures within the human vasculature including the venous anatomy make it difficult to navigate larger diameter catheters into areas of the body including the brain. For the purpose of this description, catheter systems are described with reference to venous cerebral vasculature although it is understood that the systems and methods described herein can be used in other areas of the body for treatment of conditions specific to those areas.

In a typical procedure, using available arterial cerebral access catheters, access to the cerebral venous system is obtained through femoral veins. Catheters are advanced to the inferior vena cava, through the right atria to gain access to the superior vena cava (SVA). From the SVA, access to the internal jugular vein (IVA) is achieved, followed by access to the sigmoid sinus, transverse sinus, torcula and superior sagittal sinus. Alternatively, direct access to the internal jugular vein through percutaneous puncture in the neck is also feasible.

For a larger diameter catheter, navigation from the generally more pliant neck vessels (i.e. internal jugular vein) to the contained cerebral vessels (i.e. sigmoid sinus) is the most challenging. Both pliancy of the vessels and tortuosity can create issues in the navigation of larger diameter catheters.

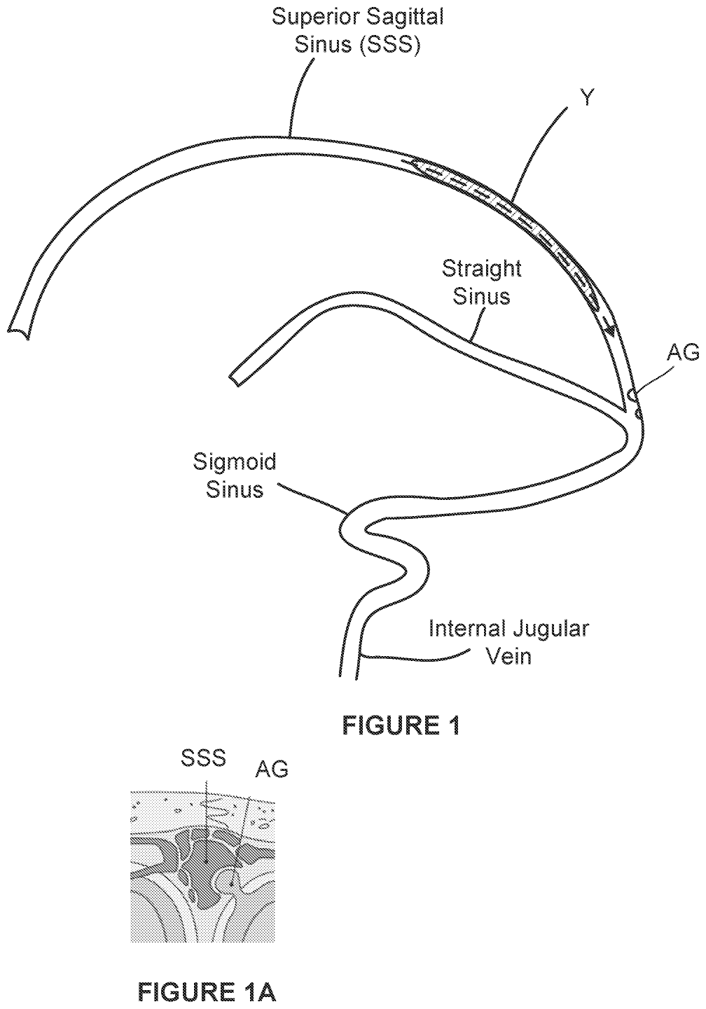

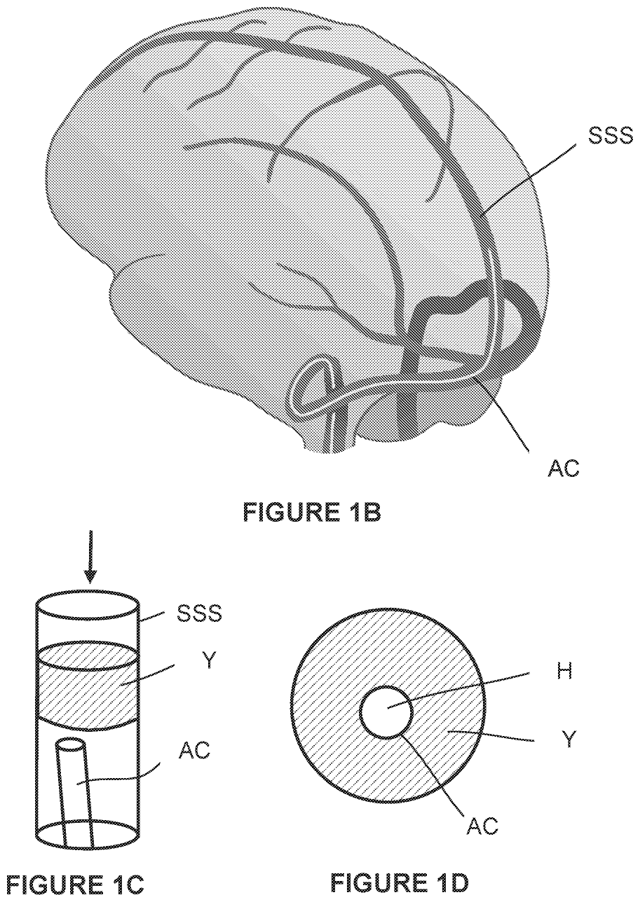

Cerebral Venous Thrombosis (CVT) is a rarer form of stroke occurring in the venous system. As shown schematically in FIG. 1, clots Y can form in situ in the cerebral venous system (e.g. Superior Sagittal Sinus) resulting in a narrowing or occlusion of the venous vessels and restricting blood flow from the brain (dotted line). These clots may be both larger in diameter/length and volume as compared to cerebral clots. During recanalization, navigation of catheter systems to the superior sagittal sinus and/or transverse sinus and/or straight sinus (common sites of dural sinus thrombosis) generally progresses from the internal jugular vein, through the sigmoid sinus and into the superior sagittal sinus. In particular, navigation from the internal jugular vein to the sigmoid sinus can be difficult due to the tortuosity and pliancy of the vessels.

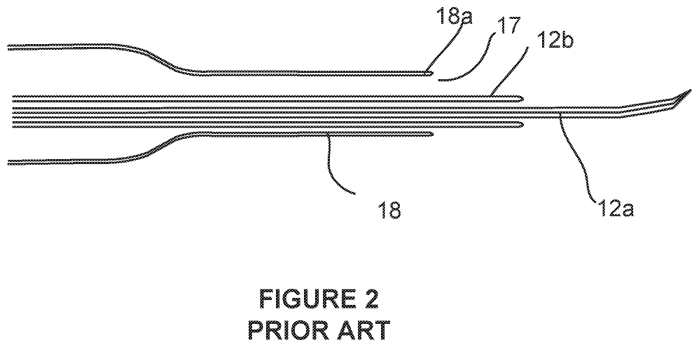

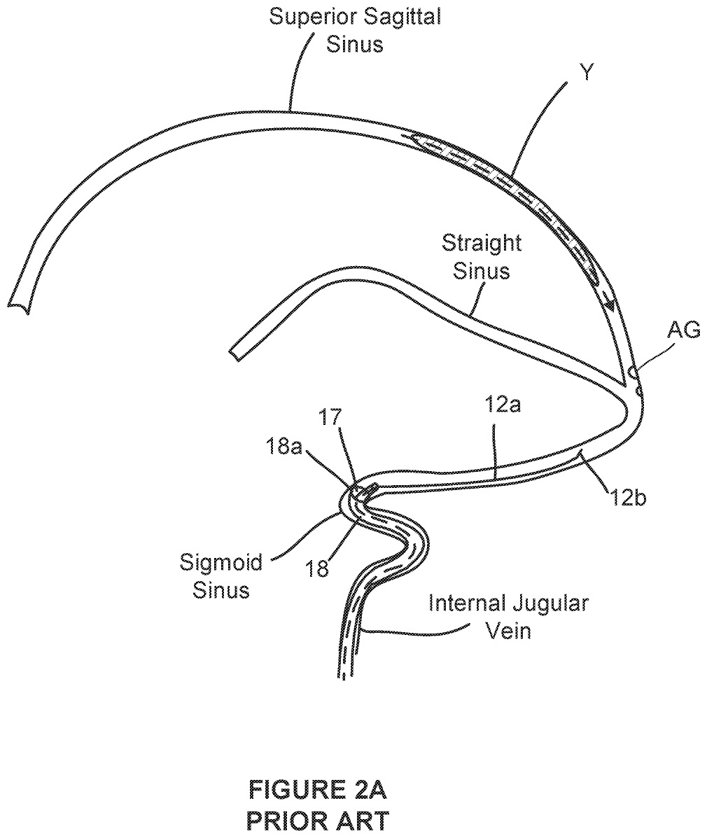

Moreover, the relative size of cerebral catheter systems compared to venous vessels can be problematic insomuch as the relatively smaller diameter and design of cerebral catheter systems can present problems as shown schematically in FIGS. 2 and 2A. Specifically, gaps 17 between the distal edge 18a of a larger catheter and smaller guide catheter can occur making it difficult to get through regions of high tortuosity and/or regions of vessel pliancy as the distal tip impacts the tight corners and can get stuck.

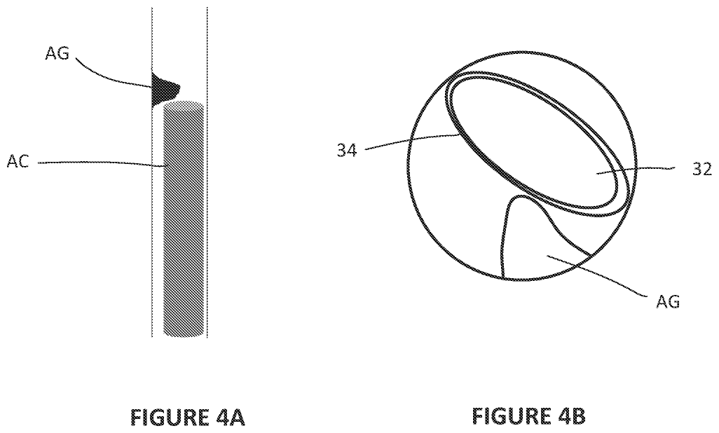

Further, the occurrence/presence of arachnoid granulations (AGs) can result in localized narrowing of vessels that create significant navigational obstructions to the advancement of catheters as shown in FIGS. 1A and 4A.

Arachnoid granulations are rigid, hard outpouchings of the dura that bulge into the cerebral veins and sinuses. Often, the inner diameter of the vein/sinus gets narrowed substantially by these AGs, with a diameter reduction of 50% or more. AGs are not compressible and thus, a catheter that would otherwise fit into the cerebral vein/sinus often gets stuck at AGs, which acts as a ledge and prevents the catheter from moving forward. Since the AG cannot be compressed by the catheter, the catheter cannot reach the site of occlusion if there is an AG proximal to the occlusion.

Large AGs are particularly common in the transverse and superior sagittal sinuses, which are the most common locations in which cerebral venous thrombosis occurs.

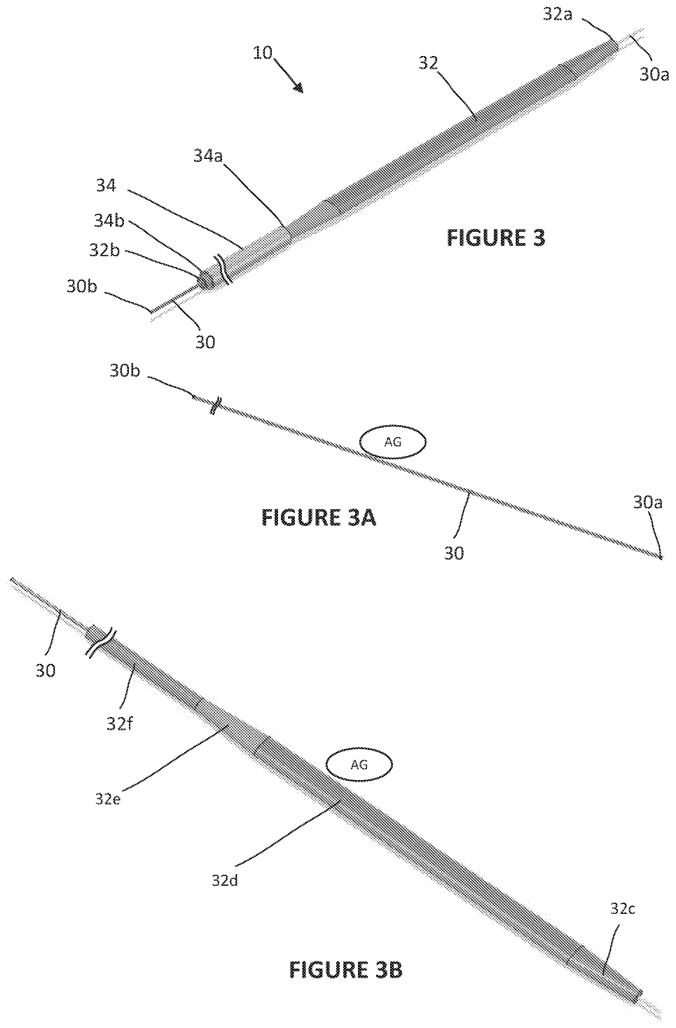

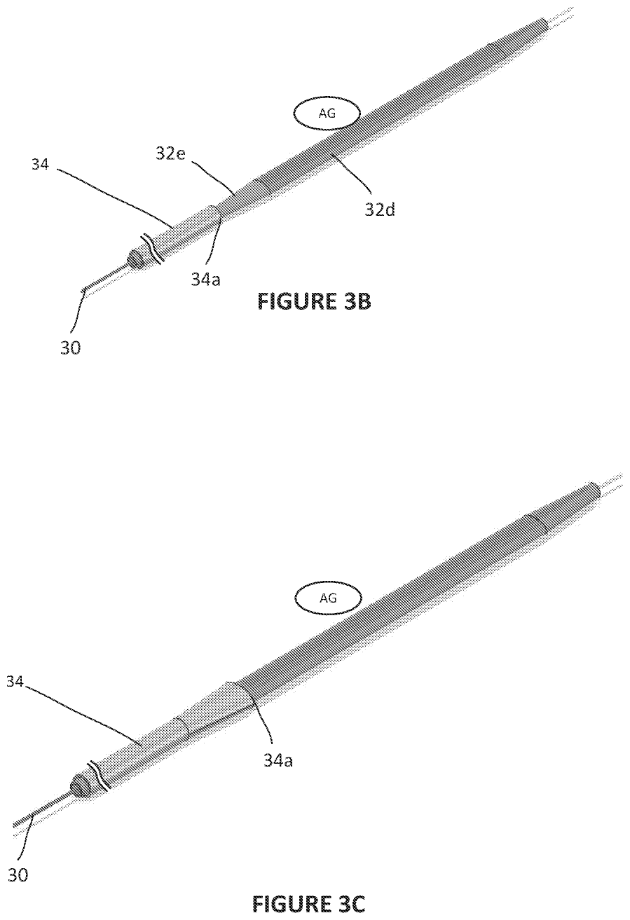

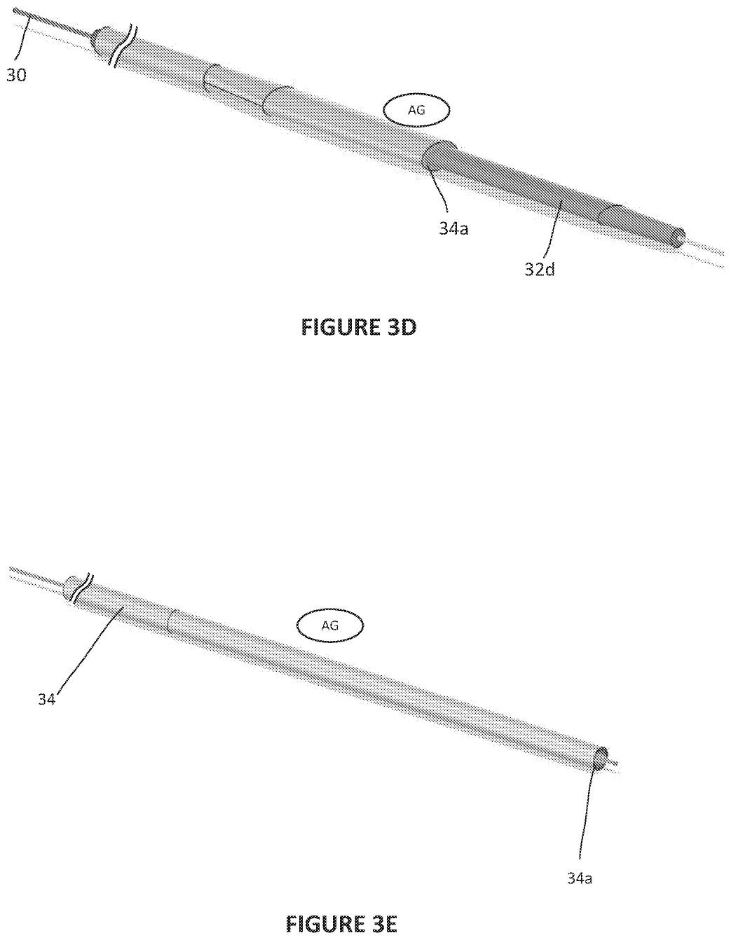

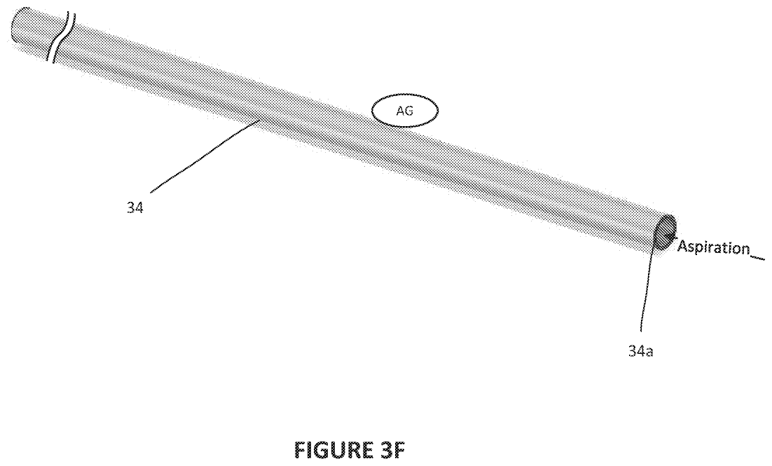

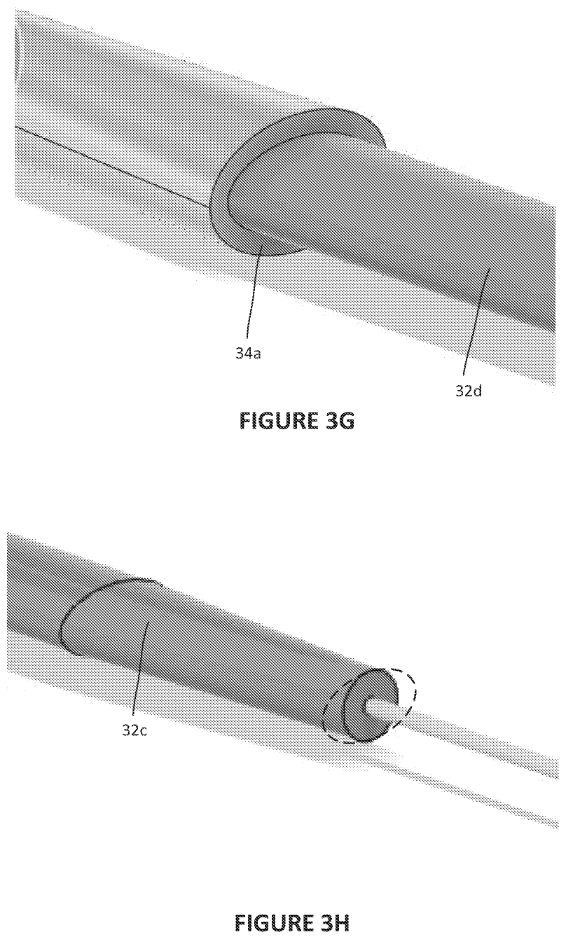

In a first embodiment, as shown in FIGS. 3, 3A-3F, the cerebral venous catheter system (CVCS) 10 includes a microwire 30, a support catheter (SC) 32 and an aspiration catheter (AC) 34. Each component includes a distal tip 30a, 32a, 34a that may be manipulated, advanced or pulled-back from a proximal end (30b, 32b, 34b).

As shown in FIG. 3B, the microwire 30 includes a distal tip 30a and proximal end 30b. The microwire is generally torquable and has a deformable or bent distal tip to enable the tip to be oriented towards a specific vessel and then advanced into the specific vessel.

As shown in FIG. 3B, the support catheter has a distal tip 32a, a proximal end 32b, a distal taper 32c, an oval section 32d, a proximal taper 32e, a proximal section 32f. The support catheter may be advanced over the guide wire.

Each of the distal taper, oval section and proximal taper are designed to support a larger bore aspiration catheter (AC) or outer catheter (OC) that may be advanced over the support catheter and wire and specifically to enable the AC to be advanced past localized narrowing of a vessel such as an AG and/or to prevent a gap 17 from forming between the AC and SC.

As shown in FIG. 4A, a localized narrowing such as an AG can create an obstruction to the advancement of a catheter. The combination of the distal taper and oval section enables both the support catheter and aspiration catheter to be advanced past a localized narrowing (e.g. an AG) by transitioning the support catheter and aspiration catheter to a flatter (e.g. oval) profile that can more effectively fill the narrow section without the distal tips of the support catheter and aspiration catheters getting stuck on the narrow section.

With reference to FIGS. 3B-3F, the system and a method of advancing the system past an AG are described. For the purposes of illustration and clarity, the vessel walls through which the system advances are not shown. An AG is shown representatively and is understood to be a narrowing as shown in FIGS. 1, 1A, 4A and 4B. It is also understood that the progression of each of the wire, support catheter and AC prior to the narrowing may be sequential, with each being advanced in an iterative sequence as known to those skilled in the art.

The following description relates to the sequence of steps to progress an AC past a narrowing and the structure of the SC in particular that enables this. For the purposes of description, the vessel is assumed to have a diameter of approximately 8 mm, the AC has an outer diameter of 6 mm (18 F), the narrowing may extend up to about 4 mm into vessel and the wire has an outer diameter of 1 mm.

As shown in FIG. 3A, a wire 30 is initially advanced past the AG. The distal tip of the wire can be readily advanced through the narrowing.

As shown in FIG. 3B, the SC 32 is advanced over the wire. The distal tip of the SC has an OD of about 2-3 mm allowing the distal tip to follow the wire past the AG. As shown, in one embodiment, the distal taper 32c transitions from a round cross-section to an oval cross-section over about 3 cm. At the proximal end of the distal taper, the support catheter has an oval cross-section having a major axis dimension of approximately 6 mm and a minor axis dimension of about 3 mm.

As the distal taper begins to engage with the AG, the distal taper may also deflect/orient itself relative to the AG such that oval section can fill the non-obstructed space as shown in FIG. 4B and be pushed past the AG. In other words, the distal taper and oval section may twist/rotate slightly in order to find the best position/orientation to move past the AG.

As shown in FIG. 3C, the AC 34 may then be pushed over the SC. The AC has a circular cross section that is larger than the cross section of the proximal portion of the support catheter.

As the AC is pushed forward, the distal tip 34a engages with the proximal taper 32e of the support catheter which, like the distal taper, transitions from the circular cross section to the oval cross section. The proximal taper stretches/distorts the distal tip 34a as the distal tip engages with the proximal taper such that the distal tip region of the AC assumes the underlying oval shape of the SC as shown in FIGS. 3D and 3E. The AC can then be pushed past the AG either by advancement of the AC and support catheter together or by sliding the AC over the support catheter.

The combined system, now past the AG, can be pushed/navigated to a desired position to initiate the procedure, such as aspiration.

Once in position, the SC and wire can be withdrawn as shown in FIGS. 3F and 3G and aspiration can be commenced. Upon withdrawing the support catheter, the distal tip 34a of the AC will return to a round shape along its length but for a possible distortion around the AG where the AC will press upon the AG.

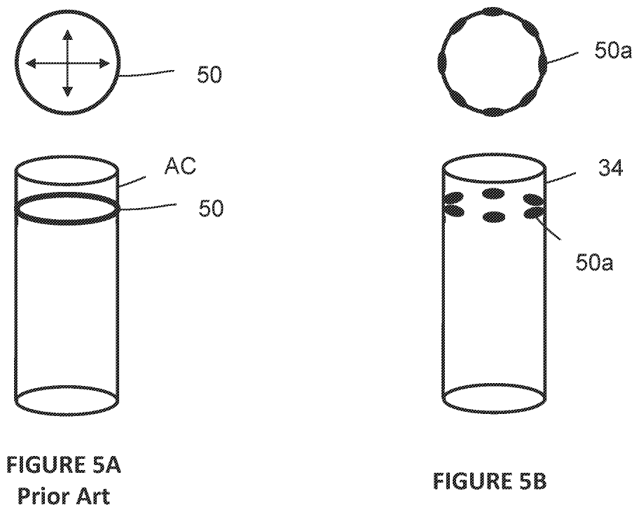

Of note, aspiration catheters normally have a radio opaque marker in the form of a metal band or ring at their distal tip as shown in FIG. 5A. This metal band increases catheter stiffness at the distal tip and may prevent the catheter tip from taking on an oval shape when slid over support catheter. That is, the ring may have a stiffness that is not readily deformed from a circular shape as shown by the arrows in FIG. 5A. Thus, in order to promote the ability of the distal tip to deform over the oval section, the radio opaque marker comprises a non-continuous marker system such as a plurality of metal dots as shown in FIG. 5B on the outer surface or buried within the distal tip with gaps that are sufficiently wide to allow the catheter tip to deform and take on an oval shape.

The SC is generally designed such that the outer perimeter length of the oval section corresponds to the inner perimeter length of the AC with sufficient tolerances that the AC can slide over the oval section. The relative ratio of lengths between the major and minor axes of the oval section are about 1.5 to 1, for example 6 mm on the major axis and 4 mm on the minor axis.

The lengths of the distal and proximal tapers will typically be about 3 cm and the total length of the oval section about 6 cm, although these dimensions can be greater or smaller as shown in Table 2.

As shown in FIG. 3H, the distal tip of the support catheter is round but, in some embodiments, could be another cross-section shape including oval (dotted lines).

Each of the wire, SC and AC typically have a total length of about 1.1-1.2 m and otherwise have sufficient length to be advanced from the femoral vein to the cerebral venous vessels. Shorter length catheters may be utilized if designed for jugular vein access or to access other parts of the vasculature.

Generally, the differences in the lengths of each of the inner and outer catheter components will be in the range of 10-20 cm. That is, the inner components will be around 10-20 cm longer than the outer components.

As described above, the distal taper and oval section of the support catheter provides support to the wider inner diameter of the AC. In addition to the ability to advance the AC past a narrowing, the system may also be used to facilitate movement through areas of high vascular curvature and/or pliancy where there is otherwise a risk that a gap 17 opens between the distal tip of the AC and the narrower sections of the support catheter.

That is, if there is an area of higher tortuosity, the AC may be advanced over the oval section to prevent a gap from opening.

In addition, it should be noted that the oval section will typically have greater flexibility in the direction of the minor axis as compared to the flexibility in the direction of the major axis. As such, in areas of tight tortuosity, the oval section will generally have a tendency to align itself such that the minor axis of the oval section is aligned towards the center of curve and provide greater bend around a tight corner.

### Methods of Use

As described above, the system may be used to access an intracranial occlusion through a patient's venous vasculature. Generally, after the surgeon has gained access to the patient's vasculature at the femoral vein, the following general steps are followed:
a. Advance the inner and outer components from the femoral vein to the inferior vena cava.
b. Advance the inner and outer components through the right atria to gain access to the superior vena cava. During steps a and b, the physician will typically be advancing these components together sequentially using the inner wire (usually of 0.035 inches) to lead.
c. Gain access to the internal jugular vein. At this stage, the physician may advance the inner components ahead of the outer components, then hold the inner components and then advance the outer components. During this step, the physician will ensure that the inner and outer components are maintained at the appropriate spacing to ensure proper engagement of the expanded surfaces.
d. This process will be continued through the sigmoid sinus, etc. to the desired location to conduct the recanalization procedure. The procedure to move past an area of tortuosity or narrowing as is described above.
e. Remove the inner components and conduct a recanalization procedure, typically by aspiration through the AC. Other adjunctive recanalization technologies (such as thrombolytic pharmaceutical agents and/or mechanical agents such as ultrasonic liquefaction of the clot) could be introduced through the OC, once the OC is in position within the clot.
f. Variations may include a tri- or quadra-axial systems with successively larger ACs with appropriate adjustments in technique.
g. Aspiration may be conducted manually via a syringe or by a mechanical pump as known for arterial side clot removal.

### CVCS Advantages

Noted advantages of this solution are:
a. If the inner components are preloaded into the distal access catheter, preparation time for surgery will be reduced.
b. The system enables larger ACs specifically designed for the venous system to be safely positioned in the larger venous vessels.
c. Larger diameter catheter allows aspiration of clot across diameter of vessel which is not possible with arterial ACs.
d. Importantly, by aspirating the venous thrombus from the patient, natural repair/cleaning processes may be triggered to reduce the clot burden. If the thrombus burden is sufficiently reduced by thrombectomy, the human intrinsic thrombolytic enzymes will dissolve the remaining thrombus remnants. Thus, reducing the thrombus burden as much as possible is desirable, even if the thrombus cannot be completely removed.
e. The system enables larger ACs to be utilized as a conduit for other treatment technologies by aspiration of the clot or introduction of other technologies or pharmaceutical agents within the clot to liquefy the clot.

In variations of the methodology, blood that is removed through the AC may be returned to the body after cleaning to remove any blood clot debris. This step is desirable given the relatively larger volumes of blood being aspirated as a result of the larger diameter vessels and larger diameter catheters.

Units of measure used in this specification are consistent with the units used in the field of endovascular surgery. That is, both imperial and metric units are used where lengths are typically expressed in metric units while diameters can be expressed in imperial units.

Key features of the cerebral venous catheter system (CVCS) are shown in Table 2.

TABLE 2
Typical Dimensions and Properties

| | |
|---|---|
| Property | Measure |

Support Catheter Components

| Overall Length | Longer (approximately 1.2 m) | |
|---|---|---|
| Distal Tip Diameter | 2 | F |
| Distal Tip Length | 10 | cm |
| Distal Taper Transition | 2-6 | cm |
| Oval Section length | 4-12 cm; preferably 4-8 cm | |
| Proximal taper transition | 2-6 | cm |
| Expanded section Diameter | 11-25 | F |

Aspiration Catheter Components

| Overall Length | Shorter (approximately 1.2 m) | |
|---|---|---|
| Distal Tip Diameter | 13-27 | F |
| Proximal Diameter | 13-27 | F |

Each of the catheters will preferably include proximal and extracorporal markings on their bodies that correspond to the relative end points of the catheters to assist the physician in understanding the relative position of each catheter to one another during a procedure.

Corresponding changes in length for systems designed for intra-jugular access can be implemented.

1. A catheter system to aid in navigating a catheter through a vessel having a narrow section or tortuous section of a patient's vasculature, comprising:
an outer catheter (OC) having an OC distal end having an OC distal end inner diameter (ID) and inner perimeter (IP);
a support catheter (SC) having an SC distal tip end, an SC proximal end and an SC expanded section adjacent the SC distal tip end, the SC expanded section having a distal taper, a proximal taper and an outer perimeter (OP), the OP substantially corresponding to the IP of the OC the expanded section for flattening the OC distal end during catheter placement.

2. The catheter system as in claim 1 where the expanded section has an oval cross-section.

3. The catheter system as in claim 1 where the OC is an aspiration catheter (AC).

4. The catheter system as in claim 1 further comprising a microwire configured for movement within the SC.

5. The catheter system as in claim 2 where SC expanded section has a major axis and minor axis and the major:minor ratio is 1.5:1.

6. The catheter system as in claim 1 where the distal taper is 4-12 cm.

7. The catheter system as in claim 1 where the proximal taper is 4-12 cm.

8. The catheter system as in claim 1 where the expanded section is 4-12 cm.

9. The catheter system as in claim 1 where the expanded section has a longitudinal axis and a distal end of the expanded section can twist about the longitudinal axis relative to a proximal end of the expanded section.

10. The system as in claim 1 further comprising one or more radio-opaque dot markers adjacent the distal tip.

11. The system as in claim 9 wherein the dot markers are at least two dot markers forming a non-continuous band around the circumference of the aspiration catheter adjacent the distal tip.

12. A catheter system to aid in navigating a catheter through a vessel having a narrow section or tortuous section of a patient's vasculature, comprising:
an outer catheter (OC) having an OC distal end having an OC distal end inner diameter (ID) and inner perimeter (IP);
a support catheter (SC) having an SC distal tip end, an SC proximal end and an SC expanded oval section adjacent the SC distal tip end, the SC expanded section having a distal taper, a proximal taper and an outer perimeter (OP), the OP substantially corresponding to the IP of the OC the expanded section for flattening the OC distal end during catheter placement; and,
one or more radio-opaque dot markers adjacent the distal tip.

13. The catheter system as in claim 12 where the distal taper is 4-12 cm.

14. The catheter system as in claim 12 where the proximal taper is 4-12 cm.

15. The catheter system as in claim 12 where the expanded section is 4-12 cm.

16. A method of advancing a catheter system through vessels of a patient having a narrowed section with the catheter system of claim 2 comprising the steps of:
a) introducing the catheter system into the patient at an access point;
b) advancing an SC and OC from the access point towards the narrowed section;
c) engaging the OC over the expanded section to distort the OC distal tip to an oval shape and pushing the supported OC distal tip past the narrowing.

17. The method as in claim 16 further comprising the step of collecting and cleaning recovered blood and reintroducing recovered and cleaned blood back to the patient.
